(19) United States
(12) Patent Application Publication
(10) Pub. No.: US 2019/0358351 A1
(43) Pub. Date: Nov. 28, 2019

(54) 177-LU LABELED ACTIVE SITE INHIBITED FACTOR VII

(71) Applicant: MINERVA IMAGING APS, Copenhagen N (DK)

(72) Inventors: Andreas KJAER, Frederiksberg (DK); Carsten HAAGEN NIELSEN, Copenhagen (DK)

(21) Appl. No.: 16/461,723

(22) PCT Filed: Nov. 17, 2017
(86) PCT No.: PCT/DK2017/050381

§ 371 Date: May 16, 2019

(30) Foreign Application Priority Data
Nov. 17, 2016 (DK) PA 2016 70913

(51) Int. Cl. A61K 51/08; A61P 35/00

(52) U.S. Cl. CPC A61K 51/088; A61P 35/00

(57) ABSTRACT

There is provided a 177-Lu labelled peptide for site-specific targeting of TF thereby enabling treatment of a cancer disease associated with high TF expression; e.g. treatment of cancer by administering to a patient an effective amount of the 177-Lu labelled peptide.

### FIELD OF THE INVENTION

The present invention relates to a ¹⁷⁷Lu labeled agent for targeted radionuclide therapy of tissue factor expressing cancers. More specifically the invention relates to the treatment of tissue factor positive tumors.

### BACKGROUND OF THE INVENTION

Various radio-labelled peptide compositions have been developed or are under development for site-specific targeting of a therapeutic radionuclide. The general principle involves attaching a selected radionuclide to a peptide having a high specificity for a particular organ or tissue so that the organ or tissue can be treated by a therapeutic radioisotope. This field of research has shown particular applicability for tumor imaging and treatment. Particularly desirable biological sites include but is not limited to neuroendocrine tumors, small cell lung carcinomas, brain tumors, prostate tumors, breast tumors, colon tumors, and ovarian tumors.

¹⁷⁷Lu-labeled peptides for nuclide targeting therapy are successfully being introduced in treatment of neuroendocrine tumors and several new targets are currently being evaluated in pre-clinical cancer models including integrins, Her-2, Gastrin-releasing peptide (GRP), and vascular endothelial growth factor (VEGF).

Tissue factor (TF) is a 47 kDa transmembrane protein, which binds factor VII (FVII) with high affinity. The resulting complex initiates the extrinsic coagulation cascade essential for normal hemostasis. Upon binding to TF, the zymogen FVII gets activated to the serine protease, FVIIa; and the TF:FVIIa complex further activates factor X eventually leading to thrombin generation and hemostasis.

In addition to its role in coagulation, TF plays a central role in cancer progression, angiogenesis, invasion and hematogeneous metastatic dissemination. Many tumors express various levels of cells surface TF, and the TF:FVIIa complex has been shown to activate protease activated receptor 2 (PAR2), and through intracellular signaling to induce an anti-apoptotic effect as well as to enhance tumor growth, migration and angiogenesis. In addition, TF:FVIIa more indirectly facilitates metastatic dissemination through thrombin generation and PAR1 signaling (1-4).

Clinically, TF is overexpressed in a number of cancers including glioma, breast, colorectal, prostate, and pancreatic cancer (5-8). Within breast, gastric, esophageal, liver, colorectal and pancreatic cancer it has been shown that TF, measured by immunohistochemistry, is associated with increased metastatic disease and is a prognostic marker of poor overall survival (1).

Targeting TF has proven effective as a cancer therapy in preclinical models. Yu et al. demonstrated that silencing of TF by siRNA reduced tumor growth in a mouse model of colorectal cancer (11). Using an immunoconjugate with FVII as the binding domain, Hu et al. suppressed tumor growth in a human melanoma xenograft mouse model (12). Ngo et al. and Versteeg et al. demonstrated that anti-TF antibodies inhibited metastasis in an experimental metastasis model and suppressed tumor growth in a breast cancer model (13,14).

Targeting of TF with an antibody-drug conjugate (ADC) was recently shown to have a potent and encouraging therapeutic effect in murine cancer models, including patient-derived xenografts models (15). A non-invasive method for specific assessment of tumor TF expression status would be valuable. Such a tool would be clinically relevant for guidance of patient management and as companion diagnostics for emerging TF-targeting therapies.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis and ischemia/reperfusion. Thus, TF is now recognised as a target for therapeutic intervention in conditions associated with increased expression.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

In addition to its established role as an initiator of the coagulation process, TF was recently shown to function as a mediator of intracellular activities either by interactions of the cytoplasmic domain of TF with the cytoskeleton or by supporting the FVIIa-protease dependent signaling. Such activities may be responsible, at least partly, for the implicated role of TF in tumor development, metastasis and angiogenesis. Cellular exposure of TF activity is advantageous in a crisis of vascular damage but may be fatal when exposure is sustained as it is in these various diseased states. Thus, it is critical to regulate the expression of TF function in maintaining the health.

Radiolabelled TF agonists and/or TF antagonists may be valuable for diagnostic imaging with a gamma camera, a PET camera or a PET/CT camera, in particular for the evaluation of TF expression of tumor cells, for grading the malignancy of tumor cells known to express TF receptors, for the monitoring of tumors with TF expression during conventional chemotherapy or radiation therapy. Also TF agonists and/or TF antagonists labelled with alpha- or beta-emitting isotopes could be used for therapy, possibly with bi-specific binding to compounds with chemotherapeutic action, which may be related to the presence of TF receptors. In those cases the diagnostic imaging may be important for the evaluation of tumor response expected after therapy with TF receptor binding drugs.

Also other kinds of diseases with increased expression of surface accessible TF receptors may be observed, maybe inflammatory or auto-immune diseases, where both diagnostic and therapeutic application of radiolabelled TF agonists and/or TF antagonists may become relevant.

One example of a TF antagonist, inactivated FVII (FVIIai) is FVIIa modified in such a way that it is catalytically inactive. Thus, FVIIai is not able to catalyze the conversion of FX to FXa, or FIX to FIXa but still able to bind tightly to TF in competition with active endogenous FVIIa and thereby inhibit the TF function.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. International patent application WO 01/21661 relates to bivalent inhibitor of FVII and FXa.

Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

WO 2004/064870 discloses active site inhibited factor Vila among other factor Vila derivatives. WO 2004/064870 also mentions the possibility of conjugating such derivatives with various radionuclides, without providing a direct and unambiguous disclosure of the 177-Lu labelled peptides of the present invention.

The efficient targeting of TF demands a selective high-affinity vector that is chemically robust and stable.

### SUMMARY OF THE INVENTION

The present invention provides 177-Lu labelled peptides having high affinity for TF, high potency in a cell-binding system, and demonstrated biological stability. More specifically the invention relates to the treatment of a cancer disease associated with high TF expression.

In a first aspect the present invention relates to a 177-Lu labelled TF binding peptide conjugate, wherein the peptide is coupled to 177-Lu by a chelating agent.

The present inventors have surprisingly found that the 177-Lu labelled peptides of the present invention are stable in vivo and capable of inducing cytotoxic effects in tumors but not in the surrounding tissue. Hence, the 177-Lu labelled peptides of the present invention constitute the optimal radionuclide for therapy of small tumor lesions and/or disseminated metastatic disease. The 177-Lu labelled peptides of the present invention specifically target TF-positive cancer cells, and in particular the most aggressive (metastatic) cells.

Specifically the present invention provides a 177-Lu labelled Tissue Factor binding peptide conjugate, wherein the peptide is coupled to 177-Lu, optionally by a chelating agent. Preferably the peptide is factor Vila (FVIIa). More preferably the FVIIa of SEQ ID NO: 1 is active site inhibited factor Vila (FVIIai) and modified in such a way that it is catalytically inactive, such as having the amino acid modification comprised of Ser344, Asp242, and His193.

In a preferred aspect of the present invention the conjugate is used in the treatment of cancer, such as breast, gastric, esophageal, liver, colorectal and pancreatic cancer.

In a further aspect of the present invention there is provided a method of treatment of a cancer disease associated with high TF expression, such as breast, gastric, esophageal, liver, colorectal and pancreatic cancer, by administering to a patient a 177-Lu labelled peptide conjugate of the present invention.

### BRIEF DESCRIPTION OF THE DRAWINGS

FIG. 1 shows ¹⁷⁷Lu-FVIIai biodistribution.

FIG. 2 shows 64Cu-FVIIai and 177Lu-FVIIai as a theranostic pair. The diagnostic agent (⁶⁴Cu-NOTA-FVIIai) has a similar biodistribution, thus highlighting the potential of using ⁶⁴Cu-NOTA-FVIIai and ¹⁷⁷Lu-FVIIai as a theranostic pair.

FIG. 3 shows ¹⁷⁷Lu-FVIIai dose escalation.

FIG. 4 shows competition with non-labeled FVIIai. Blocking with FVIIai result in markedly reduced tumor accumulation measured ex vivo by biodistribution. The blocking in the tumors (arrows) is also evident on the SPECT/CT images on the right hand side.

FIG. 5 shows tumor growth delay. One treatment with 177Lu-FVIIai results in a tumor growth delay in an animal model with tissue factor positive tumors. Additional data is pending.

### DETAILED DESCRIPTION OF THE INVENTION

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. In one embodiment of the invention the variant has a total amount of amino acid substitutions and/or additions and/or deletions independently selected from the group consisting of 1, 2, 3, 4, 5, 6, 7, 8, 9, and 10. The activation of factor VII to factor Vila, involves the hydrolysis of a single peptide bond between Arg152 and Ile153, resulting in a two-chain molecule consisting of a light chain of 152 amino acid residues and a heavy chain of 254 amino acid residues held together by a single disulfide bond.

Preferably, the FVIIa of SEQ ID NO: 1 is active site inhibited factor Vila (FVIIai) and modified in such a way that it is catalytically inactive, such as having the amino acid modification comprised of Ser344, Asp242, and His193.

By “catalytically inactivated in the active site of the FVIIa polypeptide” is meant that a FVIIa inhibitor is bound to the FVIIa polypeptide and decreases or prevents the FVIIa catalyzed conversion of FX to FXa. A FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyl lysylchloromethyl ketone (TLCK); nitrophenyl-sulphonates and related compounds; heterocyclic protease inhibitors such as isocoumarines, and coumarins.

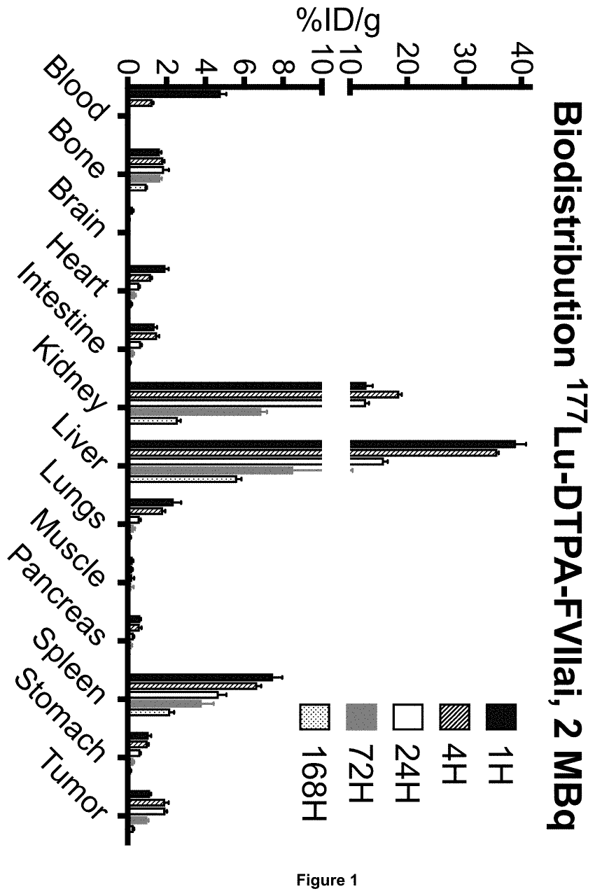

Referring to FIG. 1 there is shown ex vivo biodistribution of 177Lu-DTPA-FVIIai performed at 1, 4, 24, 72 168 and 312 hours after intra venous administration of 2 MBq 177Lu-DTPA-FVIIai in NMRI nude mice bearing subcutaneous BxPC-3 pancreatic adenocarcinoma xenograft tumors. The uptake in the tumors was 1.16±0.04, 25 1.97±0.18, 1.95±0.07, 1.01±0.06, 0.31±0.02 percent injected dose per gram (% ID/g) at 1, 4, 24, 72 and 168 hours post-injection. The delivered radiation dose is estimated to 0.12 Gy/MBq resulting in a delivered dose of 1.82 Gy to the tumors for a 15 MBq dose.

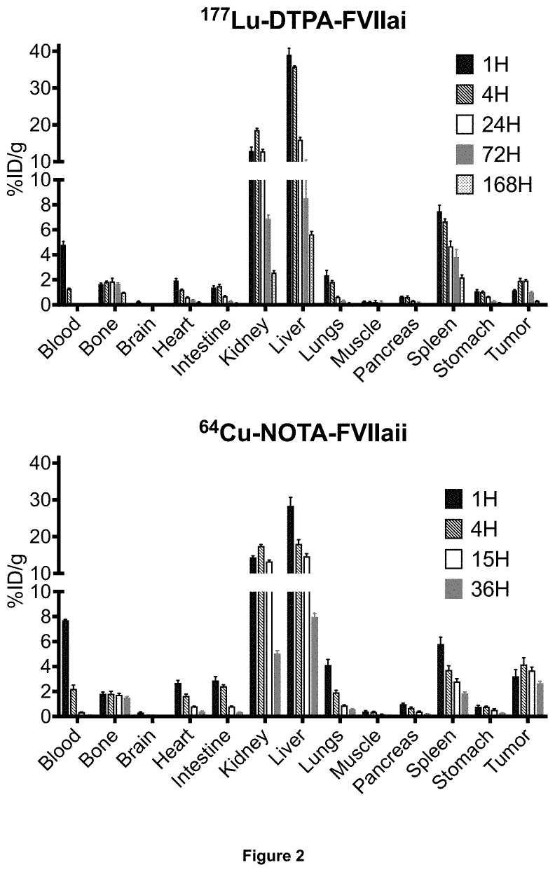

Referring to FIG. 2 there is shown a comparison of the ex vivo biodistribution of 30 177Lu-DTPA-FVIIai (top) and 64Cu-NOTA-FVIIai (bottom) shows a comparable biodistiribution. This illustrates the potential of using 64Cu-NOTA-FVIIai and 177Lu-DTPA-FVIIai as a theranostic pair for diagnostic and treatment of TF positive tumors.

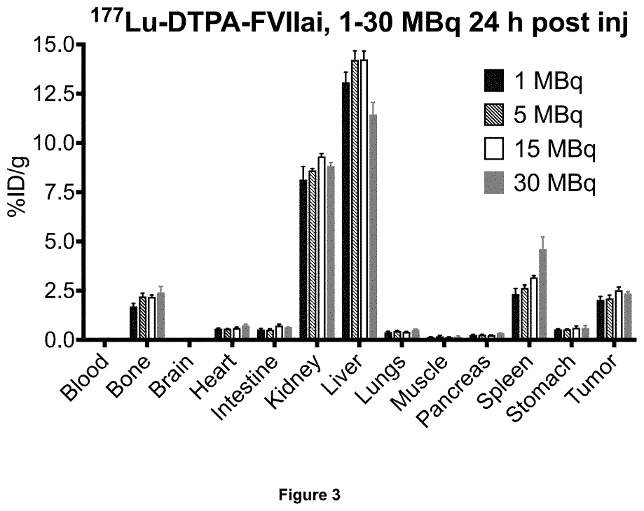

Referring to FIG. 3 there is shown the result of a dose escalation study. No saturation of the delivered fraction of 177Lu-DTPA-FVIIai is observed when increasing the dose from 1 MBq to 30 MBq per animal. This illustrates the possibility of increasing the delivered radiation dose to the tumors by increasing the injected dose.

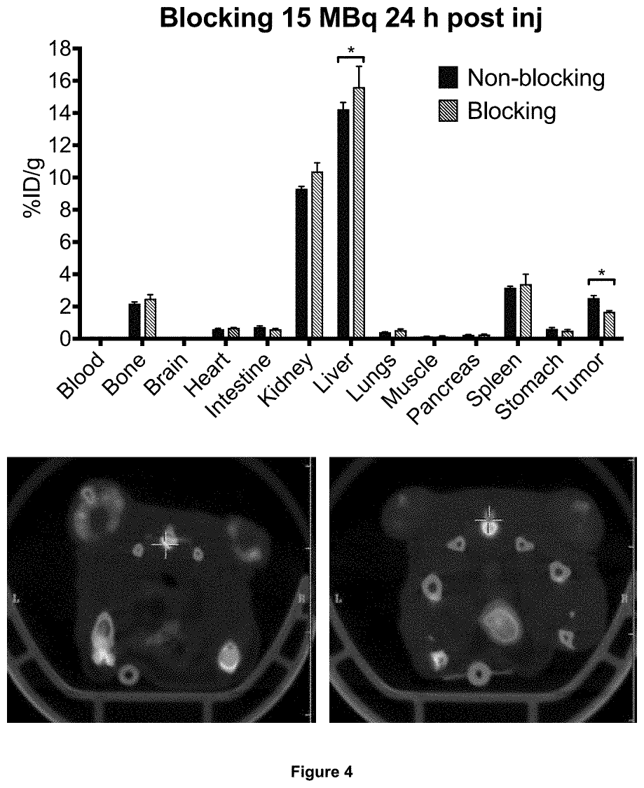

In FIG. 4 a competition study with pre-injection of an excess amount of unlabeled FVIIai was performed in mice bearing BxPC-3 tumors. The tumor uptake measured ex vivo (top) was significantly reduced in mice preinjected with FVIIai, compared to mice injected with 177Lu-DTPA-FVIIai only (2.5±0.16% ID/g to 1.7±0.05% ID/g; p<0.05). 177Lu-DTPA-FVIIai SPECT/CT imaging (bottom) of mice with (right) or without (left) pre-injection of excess FVIIai. The competition with FVIIai visually reduced the tumor uptake. Together the ex vivo and in vivo data from the competition experiment confirm the TF mediated uptake of 177Lu-DTPA-FVIIai in the tumors.

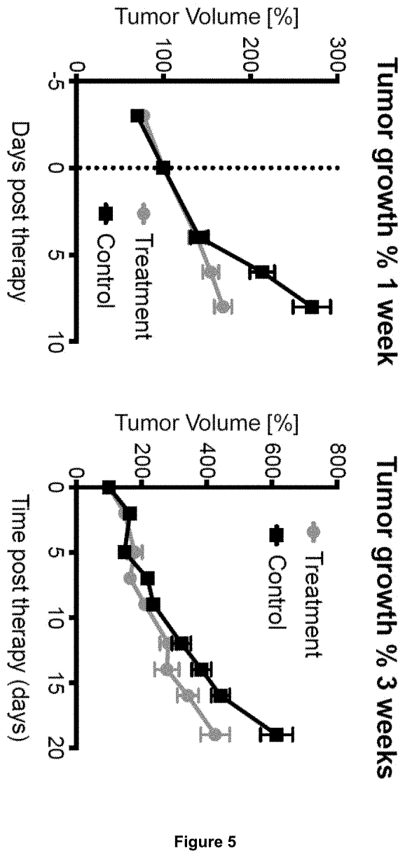

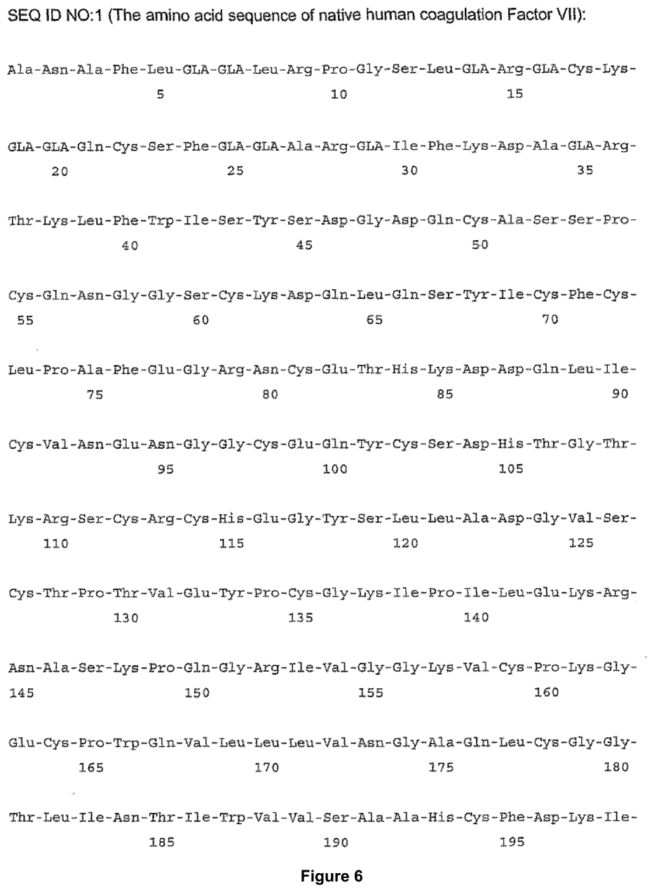

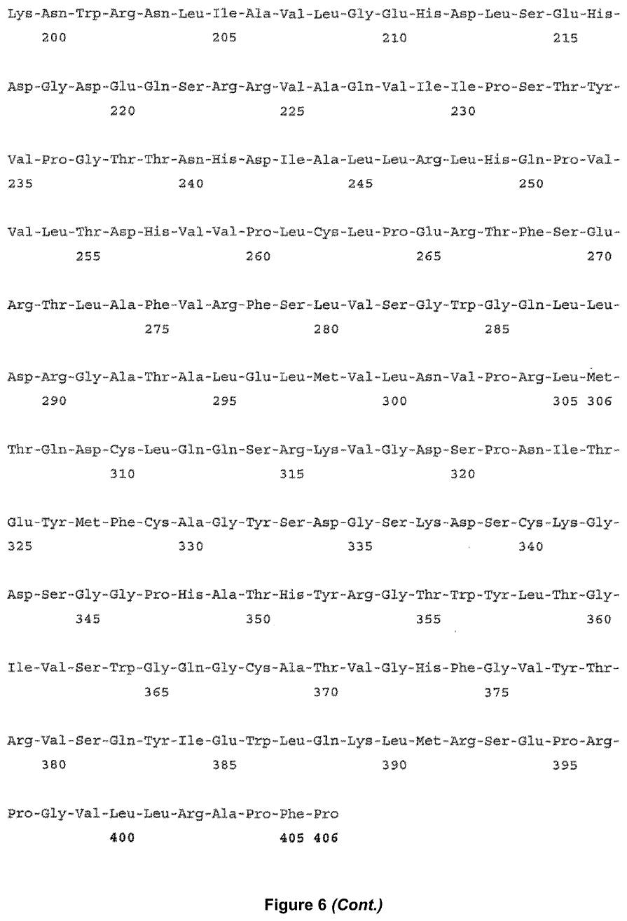

The efficacy of tissue factor targeted radionuclide therapy using 177Lu-DTPA-FVIIai is shown in FIG. 5. The effect of 177Lu-DTPA-FVIIai on the growth of subcutaneous BxPC-3 tumors was evaluated until day 8 in a pilot study (top). A significant inhibition of tumor growth was observed on day 8 (169±10% versus 270±21%; p=0.0002). The long term effect of a single injection of 15 MBq 177Lu-DTPA-FVIIai on BxPC-3 tumor growth was evaluated in a separate experiment (bottom). A significant inhibition of tumor growth was observed on day 19 (426±45% versus 614±49%; p=0.02).

1. A 177-Lu labelled Tissue Factor binding peptide conjugate, wherein the peptide is coupled to 177-Lu by a chelating agent selected from the group consisting of DTPA, DOTA, NOTA, NODAGA, CB-TE2A, H4octapa, or p-SCN-Bn-H4octapa.

2. The 177-Lu labelled peptide conjugate of claim 1, wherein the peptide is factor VIIa (FVIIa).

3. The 177-Lu labelled peptide conjugate of claim 1, wherein the FVIIa of SEQ ID NO: 1 is active site inhibited factor VIIa (FVIIai) and modified in such a way that it is catalytically inactive, such as having the amino acid modification comprised of Ser344, Asp242, and His193.

4. A 177-Lu labelled peptide conjugate of claim 1 for use as a medicament.

5. A 177-Lu labelled peptide conjugate of claim 1 for use in the treatment of cancer.

6. A 177-Lu labelled peptide conjugate of claim 1 for use in the treatment of breast, gastric, esophageal, liver, colorectal and pancreatic cancer.

7. The method of treatment of a cancer disease associated with high TF expression by administering to a patient a 177-Lu labelled peptide conjugate of claim 1.

8. The method according to claim 7, wherein the cancer disease is selected from breast, gastric, esophageal, liver, colorectal and pancreatic cancer.


SEQUENCE LISTING (1 sequence)

SEQ ID NO: 1 - protein, 406 aa, Homo sapiens [sequence omitted]